(19) United States
(12) Patent Application Publication
(10) Pub. No.: US 2017/0128130 A1
(43) Pub. Date: May 11, 2017

(54) Treatment Apparatus With a Light Guide

(71) Applicant: SEB S.A., Ecully (FR)

(72) Inventors: Camille Giraud, Lyon (FR); Thierry Gailhard, Chaponnay (FR)

(73) Assignee: SEB S.A., Ecully (FR)

(21) Appl. No.: 15/127,454

(22) PCT Filed: Mar. 19, 2015
(86) PCT No.: PCT/FR2015/050682

§ 371 Date: Sep. 20, 2016

(30) Foreign Application Priority Data
Mar. 21, 2014 (FR) 1452417

(51) Int. Cl. A61B 18/18; A61H 9/00; A61H 1/00; A61N 5/06

(52) U.S. Cl. CPC A61B 18/18; A61N 5/0616; A61H 9/0057; A61H 1/008; A61N 2005/0652; A61N 2005/063; A61N 2005/0644; A61N 2005/0666; A61B 2018/1807; A61B 2018/00452

(57) ABSTRACT

Provided is an apparatus (1) for treating skin, including: a body (2) including a casing (21, 22) forming a grippable region; electrical means for supplying power, arranged in said body; at least one treatment head (3); at least one light source (4); and at least one light guide (5) having a projection area (51) that is intended to receive the light emitted by the at least one light source (4) and to project at least one light beam intended for a region of skin. The at least one light guide (5) comprises a face opposite said projection area (51), the opposite face comprising distributing means intended to distribute the light over the projection area (51).

This invention pertains to an apparatus for treating the skin, in particular the skin of the face, designed to perform treatments of various kinds to produce a radiance-boosting, sculpting, anti-aging or anti-wrinkle effect. The treatments are administered through the application of light energy to the skin, and potentially also through a mechanical action by a means of massage.

As described in patent document KR20090001911, there already exists a massage apparatus comprising a treatment head that comprises a low-pressure chamber designed to suction the skin. The chamber includes two parallel rollers that are motorized so as to spin in the same direction. The apparatus also comprises a light emitter in the bottom of the chamber behind said rollers. The light source is of the light-emitting diode type, commonly known as LED, designed to project light simultaneously with the movement of the rollers. The disadvantage of such an arrangement of the light source is that it is far from the area being projected upon, or in other words, from the plane tangent to the exterior of the rollers, the result of which is that the intensity of the projected light is diminished. Furthermore, a portion of the light beams is hidden by the two rollers, thereby reducing the efficacy of the light treatment.

As described in another patent document, JP3155121, there already exists a portable massage apparatus comprising a treatment disc that is spun by a motor output shaft designed to come into contact with the skin. The apparatus comprises, below said disc, multiple light sources in LED form, designed to illuminate the skin through said disc, which is made of a translucent material. The light projection treatment and the disc movement treatment are performed simultaneously, in order to attain a better result. According to this document, the LEDs are arranged in a circle facing the treatment disc, in order to emit beams of light perpendicular to the skin. Nevertheless, such an arrangement does not allow for even projection or requires a great number of LEDs in order to be able to cover the entire projection surface, which increases the cost of the apparatus. Moreover, the light energy yield is not optimized, as significant energy is lost when the light passes through the translucent disc.

The objective of this invention is to remedy, at least in part, the aforementioned disadvantages and to provide a skin treatment apparatus that performs at least one mechanical treatment combined with a light treatment over a defined area.

Another objective of the invention is a skin treatment apparatus that can diffuse light evenly over the area.

Another objective of the invention is a skin treatment apparatus that is capable of producing intense, effective illumination over the area.

Another objective of the invention is a skin treatment apparatus in which the light sources can illuminate at least a portion of an area engaged by a means of mechanical massage to which they are connected.

Another objective of the invention is a skin treatment apparatus with an arrangement of light sources and massage means optimized for a smaller-sized apparatus.

Another objective of the invention is a multifunctional skin treatment apparatus that is easy to use and inexpensive.

These objectives are achieved with an apparatus for treating skin comprising a body comprising a casing forming a graspable area, a means of supplying electrical power arranged inside said body, at least one treatment head, at least one light source, at least one light guide with a projection surface designed to receive the light emitted by said at least one light source and to project at least one light beam toward an area of skin. According to the invention, said at least one light guide comprises a surface opposite said projection surface, said opposite surface comprising a distribution means designed to distribute the light over said projection surface. It is understood that the light guide is made as a single piece having both the projection surface and the opposite surface that is facing it, which decreases the number of parts and reduces the production cost, while maintaining effective light concentration. Depending on the different embodiments of the invention, the treatment head is designed to perform treatments by illumination and/or mechanical action (massage).

Advantageously, said distribution means comprises at least one surface (S1, S2) that is at least partially reflective, designed to reflect a portion of the light toward said projection surface. The at-least-partially-reflective surface is part of the opposite surface and can reflect at least a portion of the projected light, the reflection occurring inside the light guide.

In order to define its geometric shape, said at least one light guide has at least one cross-section (S) through which light passes, which is a cross-section passing through a plane (P) that is perpendicular to said projection surface, said cross-section (S) being secant to the projection surface, and said at least one surface (S1), following a first straight line (D1) and a second straight line (D2), respectively.

Thus, said at least one light source is arranged at one end of said cross-section (S) between the first straight line (D1) and the second straight line (D2). Thus, the light passes through one wall of the light guide arranged between the projection surface and the opposite surface.

In one variation of the light guide, the absolute distance between the first straight line (D1) and the second straight line (D2) is constant, measured from the end where said at least one light source is arranged. This is a light guide in the form of a generally flat plate.

In another variation of the light guide, the absolute distance between the first straight line (D1) and the second straight line (D2) decreases, measured from the end where said at least one light source is arranged. The reflective surfaces are thus arranged successively on a slope, each surface receiving at least a portion of the light emitted due to the reduction of the cross-section to the right of each surface, the further away they are from the light source.

In addition, said means of distribution comprises at least two surfaces (S1, S2) that are at least partially reflective, in which one of said two surfaces (S2) and said cross-section (S) are secant along a third straight line (D3), the distance between D1 and D2 being greater than the distance between D1 and D3. Such a passageway cross-section corresponds to a light guide with striations in the form of regular grooves on the opposite surface. This makes it possible to increase the reflection of light and decrease refraction on the opposite surface. A more even diffusion of light is obtained over the projection surface.

In a first embodiment of the apparatus, said light guide is a solid of revolution of said cross-section around a central axis (Δ). The light guide is thus circular in shape.

Advantageously, the opposite surface is fluted starting from said central axis (Δ). This makes it possible to increase the reflection of light and decrease refraction on the opposite surface. The light beams are well distributed over the circular projection surface, particularly in the areas between two neighboring light sources.

Alternatively, the opposite surface has hemispherical dimples, like a golf ball, for example, or something similar. This makes it possible to vary the angle of macroscopic reflection of the light beams and to make them even.

In addition, said opposite surface is at least partially covered in a white-colored layer. Or alternatively, said opposite surface is at least partially covered in a metallic-colored layer. This makes it possible to increase reflection and decrease refraction on the opposite surface.

Advantageously, said projection surface has a means of diffusion such as texturing, like chemical or Charmilles texturing, for example. This accentuates the diffusion of light and makes the beams more even on the skin.

Advantageously, said at least one light source is a light-emitting diode (LED), as such a light source is both intense and compact, and therefore simultaneously optimizes the efficacy and size of the apparatus.

In a second embodiment of the apparatus, said at least one treatment head comprises a means of mechanical massage designed to come into contact with the skin and a means of maneuvering said massage means powered by an electric motor that is connected to said means of electrical power supply. Performing such mechanical massages makes it possible to stimulate blood circulation in the skin of the face. The cells are further activated by the light therapy treatment applied simultaneously with the massage.

In a first variation of the second embodiment, said means of massage comprises at least one massage ball designed to be spun around at least one axis of vertical rotation that is perpendicular to the projection surface.

In this variation, said light guide has at least one through-hole through which said massage ball may pass. Thus, the light treatment can be combined with the mechanical action treatment, while maintaining the compact size of the apparatus.

Thus, said light source comprises multiple light-emitting diodes distributed at regular intervals around said light guide for even light distribution.

In a second variation of the second embodiment, said means of massage comprises a support component designed to be placed against the face, and two massage fingers designed to come into contact with the skin in an alternating manner.

In one variation, said light guide is arranged near said support component and roughly parallel to it.

Thus, said light source comprises at least one light-emitting diode between said massage fingers and the body.

In a third variation of the second embodiment, said means of massage comprises two massage rollers that can spin around one another along two horizontal axes of rotation (B1, B2) that are parallel to one another and each perpendicular to the axis of the body, a first roller comprising at least one paddle protruding radially from the surface of the first roller and the second roller having a smooth surface, said rollers being powered by a maneuvering means.

In this variation, the apparatus comprises at least one light guide designed to cover at least one contact area (Z) at the end of the rollers, designed to come into contact with the skin.

Thus, said light source comprises at least one light-emitting diode near said contact area (Z).

In a fourth variation of the second embodiment, said means of massage comprises two cylindrical rollers that can spin around one another along two axes of rotation (C1, C2) that are parallel to one another and with respect to the axis of the body.

In this variation, the apparatus comprises at least one light guide designed to cover at least one first illumination area (Z′) at one of the ends of the cylindrical rollers and/or a second illumination area (Z″) situated between said cylindrical rollers, said first illumination area (Z′) being designed to come into contact with the skin.

Thus, said light source comprises at least one light-emitting diode near said first illumination area (Z′).

Advantageously, said means of massage is transparent or translucent so as to allow light to pass through.

In addition, said at least one treatment head is detachable from said body, and the apparatus comprises at least two different treatment heads that are interchangeable on the body.

The invention will be understood more clearly upon review of the embodiments, which are in no way limiting, and which are depicted in the attached drawings, in which:

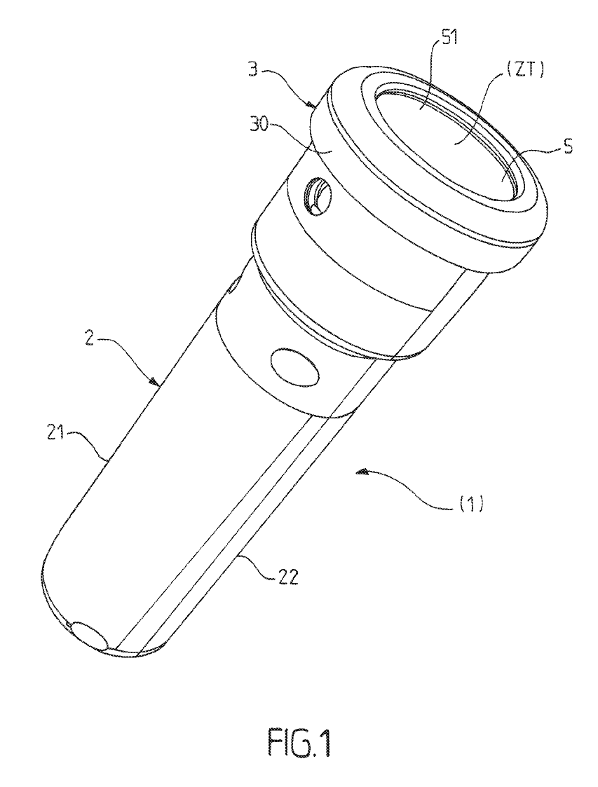

FIG. 1 depicts a view of a first embodiment of the apparatus described in the invention;

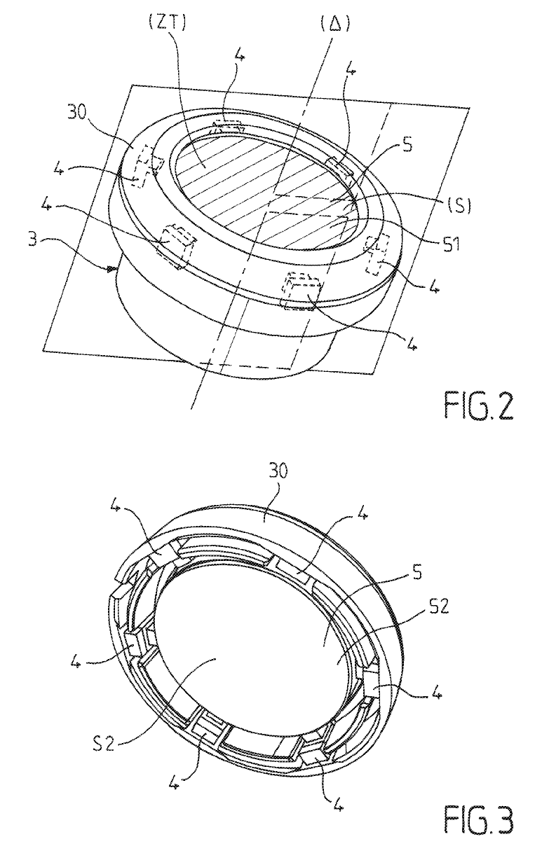

FIG. 2 depicts a view of the treatment head in the first embodiment;

FIG. 3 depicts a view of the arrangement of the light sources in the treatment head in the first embodiment;

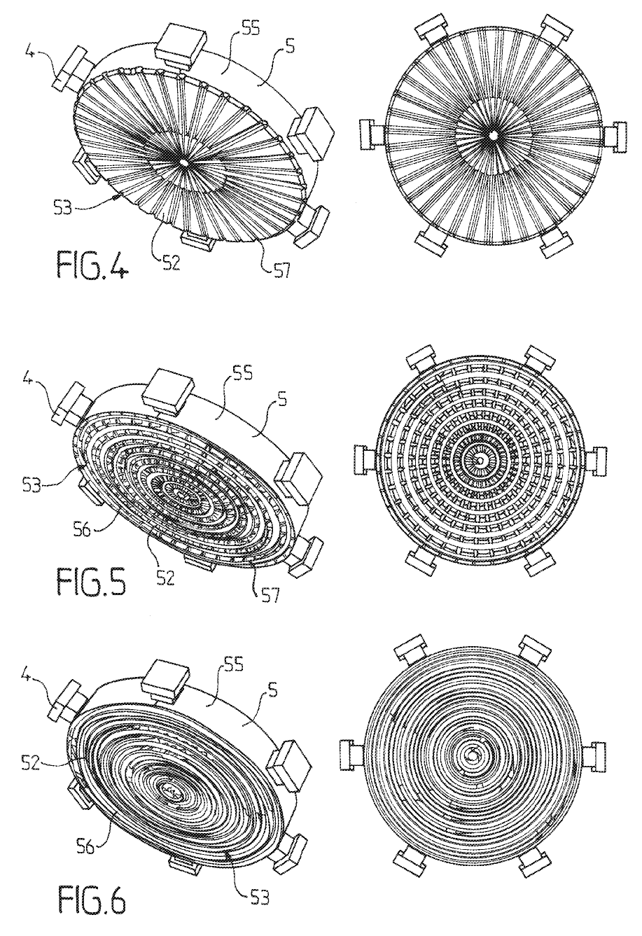

FIGS. 4 through 9 depict different variations of the light guide in the first embodiment,

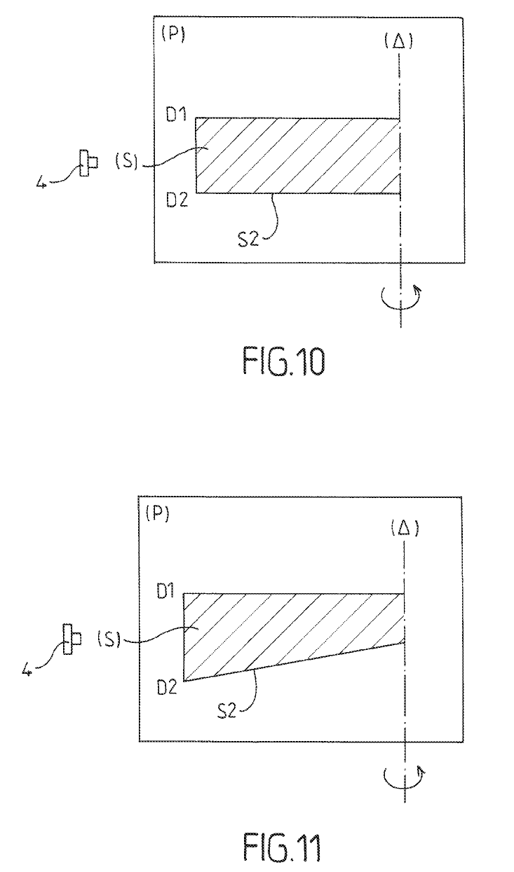

FIGS. 10 through 13 depict different light passageway cross-sections in the first embodiment;

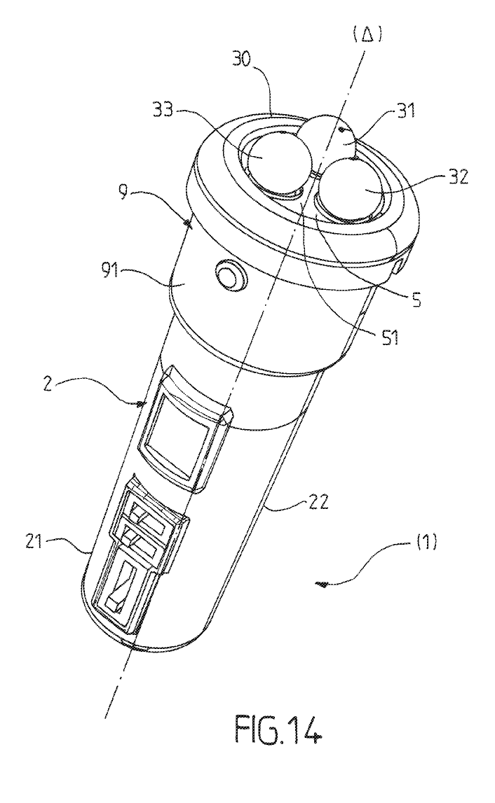

FIGS. 14 through 18 depict views of a second embodiment of the apparatus described in the invention, in a first variation;

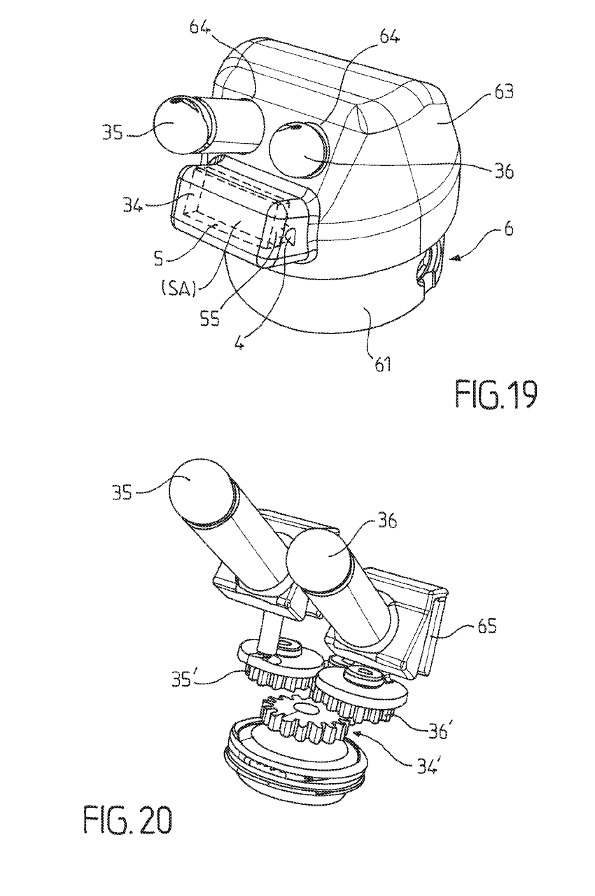

FIGS. 19 and 20 depict views of the second embodiment of the apparatus, in a second variation;

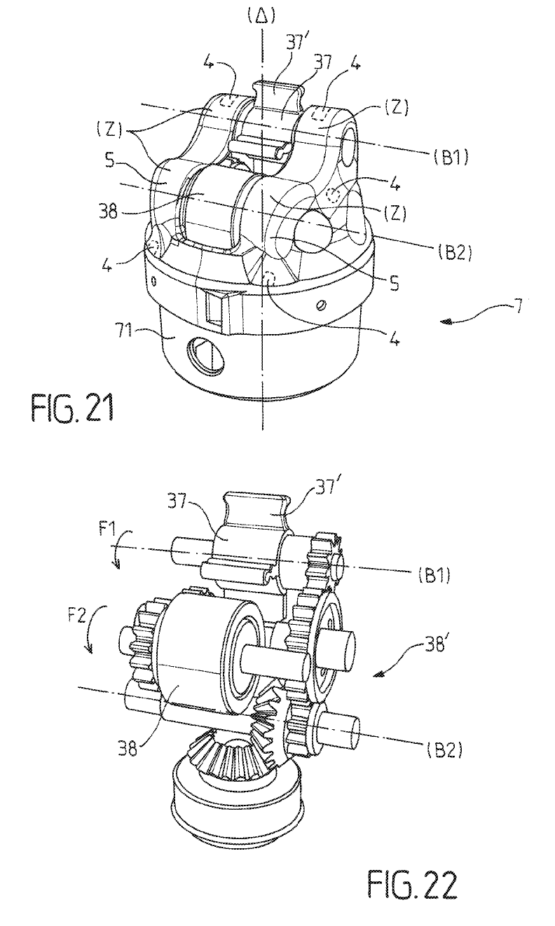

FIGS. 21 and 22 depict views of the second embodiment of the apparatus, in a third variation;

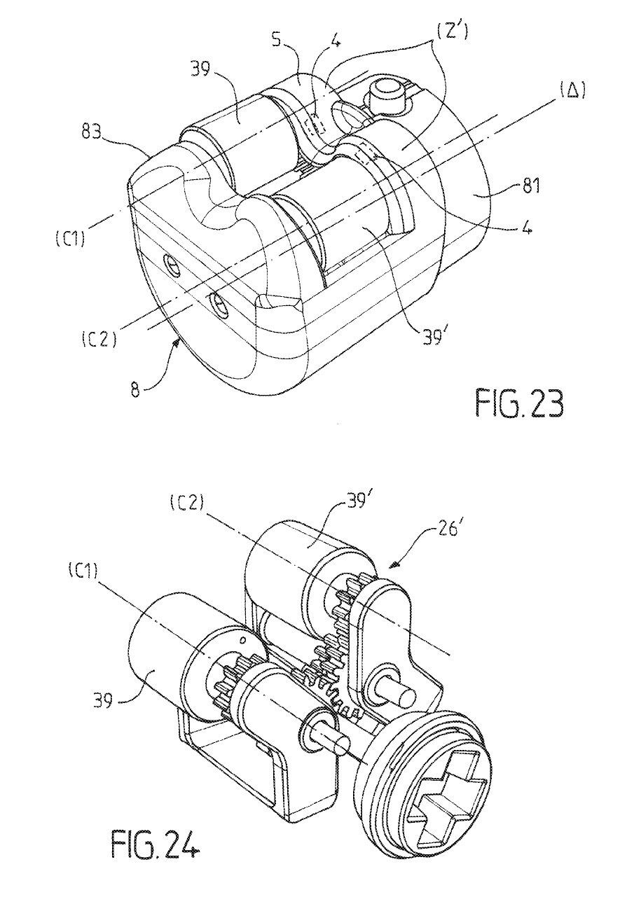

FIGS. 23 through 25 depict views of the second embodiment of the apparatus, in a fourth variation;

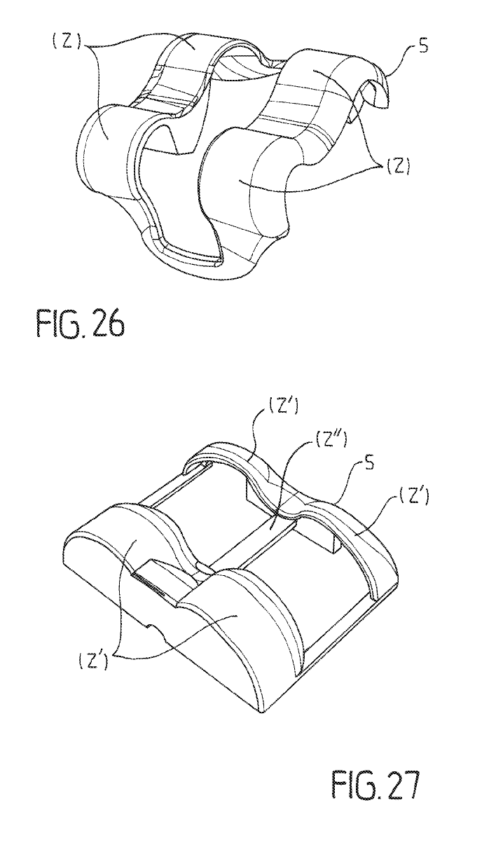

FIG. 26 depicts a view of the light guide in the third variation of the second embodiment of the apparatus;

FIG. 27 depicts a view of the light guide in the fourth variation of the second embodiment of the apparatus.

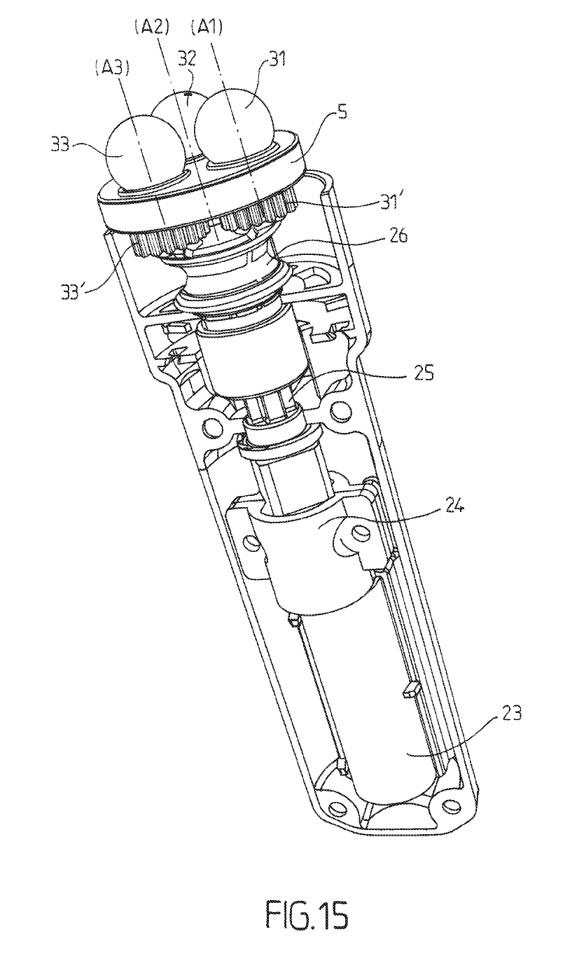

As depicted in FIG. 1, the invention pertains to an apparatus (1) for treating skin comprising a body (2) that is longitudinal in shape and has a central axis (Δ) having a casing consisting of two half-casings (21, 22) which, together, form a graspable area. A treatment head (3) is assembled on one of the ends of the body with a treatment area (ZT) designed to operate on or near the skin. Said treatment area (ZT) can be a light projection surface and/or an area with a means of mechanical massage, depending on the embodiment. The treatment apparatus (1) also comprises, inside the body (2), a means of supplying electrical power (23) as can be seen in FIG. 15, such as a rechargeable battery.

The invention will be described in two parts, with:
a first part pertaining to the first embodiment of the apparatus without a means of mechanical massage, which is illustrated in FIGS. 1 through 13;
a second part pertaining to the second embodiment of the apparatus in its different variations, the second embodiment comprising a means of mechanical massage that is depicted in FIGS. 14 through 27.

Part 1

As depicted in FIGS. 2 and 3, the treatment head (3) is substantially cylindrical in shape with a central axis (Δ) and comprises multiple light sources (4) that are LEDs arranged inside the cylinder. In the example depicted, there are six LEDs (4) distributed at regular intervals around the circumference of the cylinder. The LEDs (4) are supplied with power by the electrical power supply means (23) through electrical wires (not depicted) such that each LED is oriented toward the center of the cylinder and projects light beams in that direction. With regard to the positioning of the LEDs (4), the treatment head (3) comprises a ring (30) with circumferential holes into which the LEDS are fitted, said ring (30) being transparent, translucent or even opaque. Another purpose of the ring (30) is to conceal the LEDs so that they are protected from the exterior.

The interior of the treatment head (3) comprises a light guide (5), the purpose of which is to orient the beams of light from the LEDs toward the skin, to make them more even and to concentrate them. To this end, said light guide (5) is in the shape of a roughly circular, single-piece plate with a smooth exterior surface that is a light projection surface (51). Said projection surface (51) defines the treatment area (ZT), which is designed to be placed near the skin or even against the skin. The light guide (5) also has an opposite surface (52), which is arranged facing said projection surface (51) and is separated from it by a roughly cylindrical lateral wall (55) that can be seen in FIG. 4. The LEDs are therefore distributed around the light guide (5) facing the lateral wall (55).

Said opposite surface (52) comprises a means of distribution (53) designed to distribute the light over the projection surface (51). In a first variation of said distribution means (53), it is a smooth, reflective surface (S2) as can be seen in FIG. 3, designed to receive the light beams from the LEDs and to reflect them toward the projection surface (51). To better illustrate this point, one could imagine a cross-section (S) for the passage of light toward the center, which is obtained by the intersection of plane (P) perpendicular to said projection surface (51), the light guide (5) and the central axis (A). As depicted in FIG. 10, the cross-section (S) and the projection surface (51) are secant along a first straight line (D1); the cross-section (S) and the opposite surface (52) are secant along a second straight line (D2). The distance between D1 and D2 is constant, measured from the end where the LED is arranged. The light guide (5) in this variation is therefore a solid of revolution of said cross-section (S) around the central axis (Δ).

The distribution means (53) could also take on other different forms, which are depicted in FIGS. 4 through 9. In this second variation, the distribution means (53) comprises a series of reflective surfaces that are in the form of striations on the opposite surface (52).

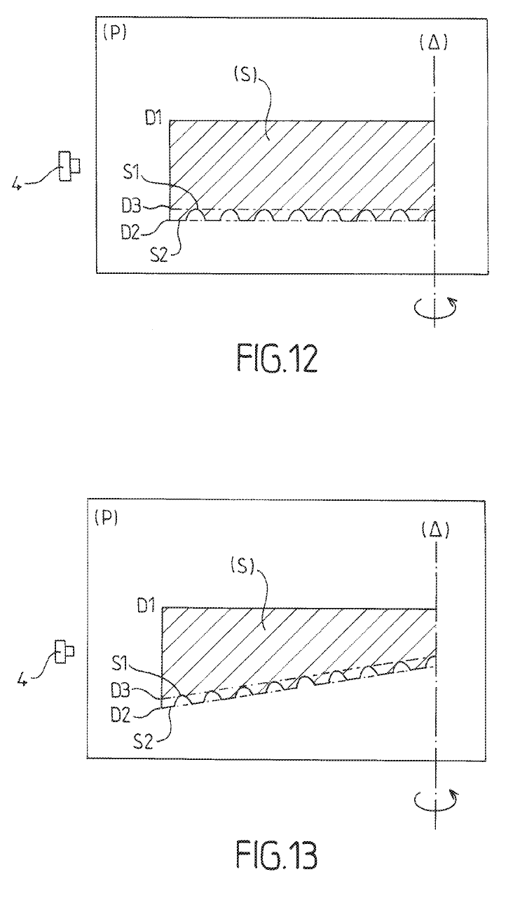

In a first variation of the striations, the distribution means (53) has, on the opposite surface (52), depressed concentric circles (56) to guide the light beams to cover the entire cross-section (S) to the center of the light guide (5), in order to have an even distribution of light over the projection surface (51). In this variation, the second straight line (D2) representing the non-depressed part of the opposite surface (52) is a dotted line. The cross-section (S) and the light guide (5) are secant along a third dotted straight line (D3) representing the depressed part of the opposite surface (52), the distance between D1 and D2 being greater than the distance between D1 and D3. FIG. 12 shows the cross-section (S) constituting the depressed circles (56) by revolution around the central axis (Δ); the variations of the light guide (5) having such circles are depicted in FIG. 6.

In a second variation of the striations, the distribution means (53) has flutes (57) on the opposite surface, radiating from the central axis (A) toward the lateral wall (55) in order to distribute the light over the projection surface (51) between two neighboring LEDs, as depicted in FIG. 4.

The depressed concentric circles (56) and the radial flutes (57) could be combined in order to have a better distribution of light. Such a combination makes it possible to vary the angle of macroscopic reflection of the light beams and to make them even. An example of the light guide (5) combining the two means of distribution is depicted in FIG. 5.

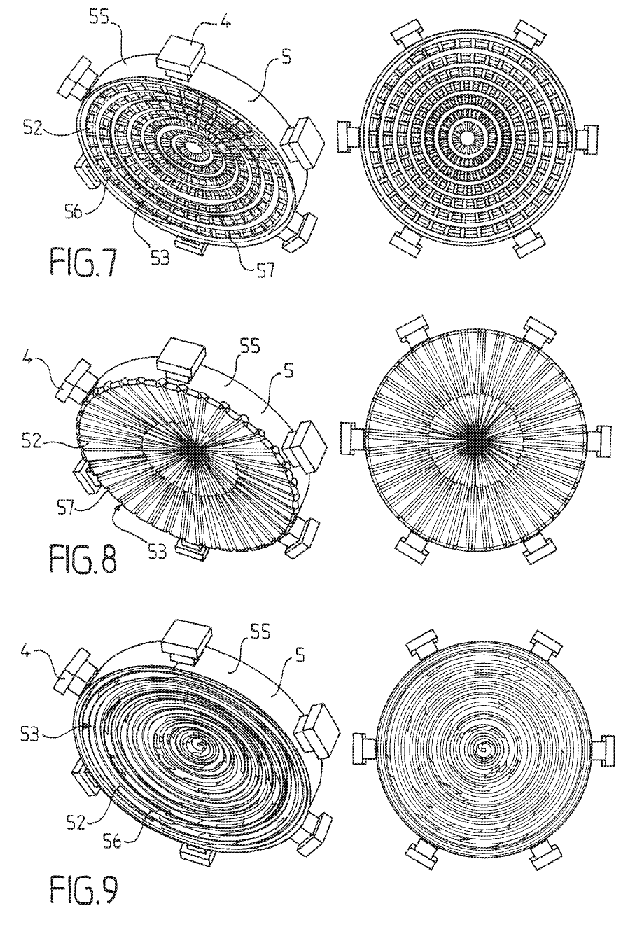

Still in reference to the example of the circular plate, the distribution means (53) can also comprise a conical surface that is depressed toward the interior of the plate. In the variations in which the means of distribution (53) is a smooth surface or a fluted surface (57), the opposite surface forms a slope, as shown in FIG. 11, so that the entire surface is used to reflect a portion of the light emitted. In the variation in which the means of distribution comprises depressed circles (56), the distance between D1 and D2 decreases, as measured from the end of the light guide (5) to the central axis (Δ) as can be seen in FIG. 13. The light guide in this variation is depicted in FIG. 9. Thus, each striation is used by the light beams to send them toward the projection surface (51). The conical shape of the opposite surface improves the way the light beams are transmitted and distributed toward the skin in order to increase the efficacy of the light, due to a stronger overall intensity. Of course, the light guide with such a conical shape on the opposite surface could only have flutes (57) as depicted in FIG. 8, or the two types of striations as can be seen in FIG. 7.

The light guide (5) can be made of a plastic material such as PC (polycarbonate), colorless PET (polyethylene terephthalate), SAN (styrene acrylonitrile) or a thermoplastic material such as PMMA (polymethyl methacrylate) which offers a good medium refractive index, good reflection to the interfaces and good transparency for reduced energy absorption and therefore reduced energy loss. Other materials could also be considered, such as glass.

In order to hide the interior of the light guide and to make the light beams project as evenly as possible, the projection surface (51) can be unpolished or textured, with a chemical or Charmilles type of surface texturing, for example.

Part II

We will now describe the second embodiment of the invention, in which the apparatus comprises a means of massage (31, 32, 33, 34, 35, 36, 37, 37′, 38, 39, 39′) according to four variations as depicted in FIGS. 14 through 27.

In the second embodiment, each treatment head (6, 7, 8, 9) comprises a means of adapting the treatment head (6, 7, 8, 9) on the body (2) in a removable manner, said means of adaptation (61, 71, 81, 91) being formed by a sheath enabling the treatment head (6, 7, 8, 9) to be partly inserted into the body (2). The body (2) comprises an electric motor (24) connected to an output shaft (25) by a means of transmission, the output shaft (25) being accessible at the end of the means of adaptation (61, 71, 81, 91). The electric motor (24) is controlled by a control unit connected to a manual control interface from the exterior of the body (2). The manual control interface may comprise, for example, a start/stop switch and/or a means of manually selecting the operating program.

The treatment head (9) in the first variation, as depicted in FIGS. 14 through 18, is generally cylindrical in shape and coaxial with the body (2) of the apparatus. The treatment head (9) comprises a support ring (30) outlining a work area inside of which are three massage balls (31, 32, 33) protruding from the plane of the ring (30), as depicted in FIG. 14. In the example depicted, each massage ball has a contact surface that can be deformed to effectively manipulate the skin with the movement of the balls. For example, the ball can be at least partially made of silicone, for a soft, grippy effect. As depicted in FIG. 15, each massage ball (31, 32, 33) is connected to a maneuvering means in order to be rotated not only with respect to the central axis (Δ) but also with respect to its own axis of vertical rotation (A1, A2, A3). To this end, the maneuvering means comprises a driver (26) designed to fit together with the output shaft (25) of the motor to initiate its movement. For example, the driver (26) may have a cross-shaped hole designed to receive the output shaft (25) by a protrusion of the same shape on the shaft. On an upper surface, the driver (26) of the axis (Δ) holds three pins (not depicted) that are each inserted in an axial bore of a planet wheel (31′, 32′, 33′). Each massage ball is attached to the corresponding planet wheel. Each planet wheel engages with the toothed perimeter (301) of a fixed band (50), depicted in FIG. 17, such that the rotation of the driver (26) drives the planet-like movement of the massage balls (31, 32, 33), or in other words a main rotation movement of the three balls around the central axis (Δ) combined with a secondary rotation movement of each of the balls around the vertical rotation axis (A1 , A2, A3), which rotates around the central axis (Δ) while the apparatus is operating.

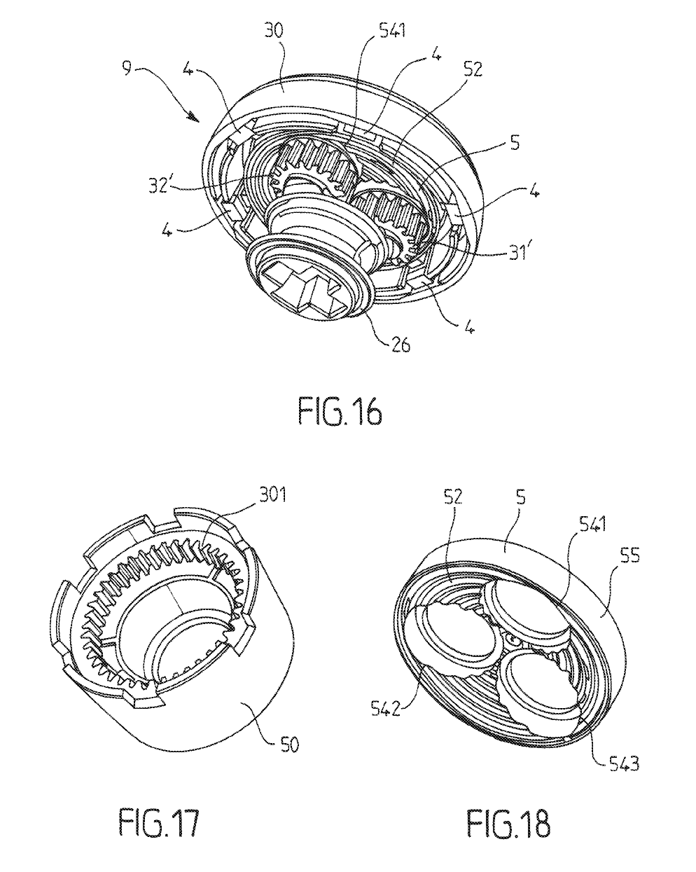

As depicted in FIGS. 16 and 17, the treatment head (9) comprises a series of LEDs arranged around the circumference of the ring (30), as in the first embodiment. The light guide (5) in this variation is arranged between the support ring (30) and the fixed band (50). As shown in FIG. 18, the light guide (5) has through-holes (541, 542, 543) designed to allow the massage balls (31, 32, 33) to pass through. The spinning of the light guide (5) is driven by the movement of the balls.

The treatment head (9) as described in this first variation makes it possible to knead the skin of the face, particularly the broad areas such as the cheeks or forehead, to activate microcirculation and stimulate the skin's natural production of structural proteins, thereby preventing signs of ageing. These effects are accentuated by the presence of light on the skin.

The treatment head (6) in a second variation, as depicted in FIGS. 19 and 20, is designed to perform a tapping massage. To this effect, the treatment head (6) comprises a support component (34) that is designed to be placed against the face to define a working distance. Thus, the support component (34) defines a support surface (SA), which forms a reference surface. The support surface (SA) is ideally smooth, and it may be concave in shape, which allows it to fit the contours of the cheekbones while the massage is being performed beneath or around the eyes.

Above the support surface (SA) and opposite the body (2) with respect to said support surface (SA), the treatment head (6) comprises massage fingers (35, 36), each of which comprises a working head designed to come into contact with the face. The two massage fingers (35, 36) are each mobile between, on the one hand, a retracted position corresponding to the position of the massage finger (36) situated in the foreground in FIG. 19, and on the other hand, an extended position corresponding to the position of the massage finger (35) situated in the background of the same figure. In the retracted position, the working head of each massage finger (35, 36) is below the support surface (SA) toward the interior of the treatment head (6). Whereas, in the extended position, the working head of each massage finger (35, 36) extends beyond the support surface (SA), toward the exterior of the treatment head (6). Thus, between its retracted and extended positions, each working head has a range of movement of between 5 mm and 15 mm. Moreover, in the extended position, each working head protrudes past the support surface (SA) by a distance of between 2 mm and 10 mm.

The treatment head (6) also comprises maneuvering means (34′, 35′, 36′) adapted to move each of the massage fingers (35, 36) in an alternating fashion between their extended and retracted positions. The maneuvering means (34′, 35′, 36′) are thus adapted to cooperate with the output shaft (25) so as to transmit and transform the rotational movement of the electric motor (24) into an alternating movement of the massage fingers (35, 36).

In the example depicted, each massage finger (35, 36) is made in the shape of a sort of rectilinear piston that extends, in part, at least to the exterior of a hollow body (63) surrounding the maneuvering means (34′, 35′, 36′). Each finger is then guided in translation by a bore (64) arranged in the hollow body (63). The end of each massage finger (35, 36) situated inside the hollow body (63) cooperates with an off-center pin (not depicted) held by a maneuvering disc (35′, 36′) belonging to the maneuvering means. The off-center pin is positioned in a chamber (65) that is connected rigidly to the corresponding massage finger and in which the off-center pin can move in translation such that its rotation with the maneuvering disc (35′, 36′) is transformed into a translation of the corresponding massage finger (35, 36).

It would be very easy to imagine other embodiments of the tapping treatment head (6), such as a crankshaft system or even propulsion by a cam that swings the massage fingers in an alternating fashion around an axis.

As depicted in FIG. 19, the treatment head (6) in the second variation comprises at least one LED (4) in the hollow body (63) and at one end of the support component (34) such that the light beams are emitted toward the opposite end. The treatment head (6) thus comprises, in the hollow body (63), a light guide (5) that is roughly rectangular and that corresponds substantially to the support surface (SA). The LED is therefore arranged at the end of the light guide (5) facing one of its lateral walls (55). The projection surface (51) is near the support surface (SA) and the opposite surface (52) can be parallel to the projection surface (51) or indeed get gradually closer to the projection surface (51) starting from the end with the LED (4).

In order to effectively illuminate the skin, the support component (34) is transparent or translucent in order to allow the light to pass through.

Such a means of massage makes it possible, through the tapping performed by the massage fingers, to stimulate blood circulation around the eyes in order to reduce dark circles and bags. The massage apparatus can also be used to boost the metabolism, particularly the production of the skin's building blocks, and thus treat wrinkles and fine lines of the face, particularly in the smile lines, by stimulating blood circulation, which is slowed by the folding of the skin in the wrinkles. These effects are accentuated by the presence of light on the skin.

The treatment head (7) in the third variation, as depicted in FIGS. 21 and 22, is designed to perform a pinching massage. To this effect, the treatment head (7) comprises, as a means of massage, two massage rollers (37, 38) that can rotate around one another along two axes of horizontal rotation (B1 and B2), which are parallel to one another and perpendicular to the central axis (Δ) as better shown in FIG. 21. The two massage rollers (37, 38) are separated from one another by a working zone. In the example depicted, the distance between the axes of horizontal rotation (B2 and B2) is constant.

Moreover, in the example depicted, the massage rollers (37, 38) are arranged inside the treatment head (7), such that a plane tangent to the two rollers and situated toward the exterior of the treatment head (7) forms, with a longitudinal axis of the body (2), a non-zero angle that is not a right angle.

According to the invention, a first roller (37) comprises at least one paddle (37′), and in this case shown as an example, four paddles that protrude radially from the surface of the first roller (37). The treatment head (7) has, on either side of the rollers (37, 38) a contact area (Z) designed to be pressed against the skin to hold the apparatus on the skin during the treatment. The peripheral surface of the first roller (37) is thus held away from the contact area (Z). The paddles (37′) extend radially enough to protrude beyond the contact area (Z) as the first roller (37) rotates. The paddles (37′) are also distributed at regular intervals on the periphery of the first roller (37) and are, in this case, positioned at 90° from one another.

In the example depicted, the paddles (37′) have cross-sections of different shapes, with the understanding that all the paddles could be the same shape. Thus, the paddle (37′), viewed as a cross-section, is shaped like a figure-eight. Other paddle shapes could also be considered, such as a paddle that, when viewed as a cross-section, has a free end that is thicker than the rest, or a paddle that, when viewed as a cross-section, is straight in shape, or finally, a paddle that, when viewed as a cross-section, is long and slender in shape and gets thinner toward the end.

The second roller (38) has a smooth peripheral surface that protrudes from the contact area (Z) or that extends alongside the contact area (Z).

According to the invention, the treatment head (7) also comprises a maneuvering means (38′) adapted to drive the rollers (37 and 38) in the same direction, going from the exterior of the working zone toward the interior of the working zone for the first roller (37), and from the interior toward the exterior of the working zone for the second roller, when viewed from the exterior of the treatment head, and as indicated by Arrows F1 and F2 in FIG. 22. The maneuvering means (38′) are thus adapted to cooperate with the output shaft (25) so as to transmit and transform the rotational movement of the electric motor along the central axis (Δ) into rotational movements along Axes B1 and B2, the directions of which are orthogonal to that of the central axis (Δ).

According to the example depicted in FIG. 22, the maneuvering means (38′) comprises a gear train (38′) comprising, first, two truncated conical gearwheels that provide angle transmission and, next, straight gearwheels that drive the massage rollers (37 and 38) simultaneously, but at different speeds. The maneuvering means (38′) are preferably adapted to ensure a rotation speed of the first roller (37) that is greater than that of the second roller (38) and in this case, triple that of the second roller (38).

Moreover in this variation, the treatment head (7) comprises LEDs near the contact areas (Z). Several arrangements of the LEDs could be considered, given that the contact areas (Z) are each covered by a light guide (5). In the example depicted, two LEDs are arranged at the top of the treatment head (7), or in other words, at the end of the contact areas (Z) away from the body (2); two other LEDs are arranged at the bottom of the treatment head (7), or in other words at the other end of the contact areas (Z). The light is thus transmitted by the light guide (5), which is shown in FIG. 26. The light guide according to this example is in the shape of a single-piece hull with a roughly constant thickness, conforming to the shape of the contact area (Z). One could also consider an arrangement of LEDs in the middle of the contact areas (Z) to increase the illumination between the two rollers. The light guide could thus be thicker in the middle than at its ends.

Such a massage means makes it possible to massage the skin by light pinching so as to replicate the “Jacquet pinching” type of massage performed by professional estheticians, which consists of using the thumb and index finger for a delicate massage. These effects are accentuated by the presence of light on the skin. The treatment head (8) in the fourth variation, as depicted in FIGS. 23 through 25, is designed to perform a “palpate and roll” type of massage on the skin to achieve a sculpting effect. Such a treatment head (8) comprises a roller chamber (83) with an application surface (Z′) designed to press against the skin and two cylindrical rollers (39, 39′), a portion of which extends beyond the application surface (Z′). These cylindrical rollers (39, 39′) are positioned along two axes (C1, C2), which are parallel to one another and with respect to the central axis (Δ). As shown in FIG. 24, the treatment head (8) comprises a maneuvering means (26′) comprising a disc spun by the output shaft (25), the disc being connected in rotation to a toothed wheel. The maneuvering means (26′) also comprises a gear train connecting the toothed wheel to two gearwheels connected in rotation to each of the two cylindrical rollers (39, 39′), respectively, and designed to drive the rollers.

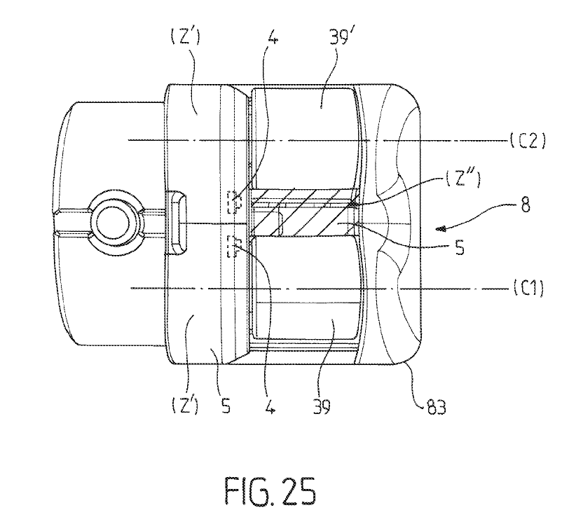

In the invention and as depicted in FIGS. 23 and 25, the treatment head (8) also comprises LEDs (4) arranged at one of the ends of the cylindrical rollers (39, 39′), preferably at the end closer to the body (2) of the apparatus. In order to better project the light from the LEDs, the treatment head (8) is equipped with a light guide (5) in the form of a single-piece hull shaped like an “I” when viewed from the front. The light guide (5) thus comprises at least a first part covering said application surface (Z′) to form a first illumination area (Z′) and a second part covering a surface at the bottom of the roller chamber (83), particularly the one between the two cylindrical rollers (39, 39′) to form a second illumination area (Z″). The light guide (5) according to this example is depicted in FIG. 27.

Such a massage means makes it possible to optimize the working of the skin through folding, in a complete, pleasant, effective manner. These effects are accentuated by the presence of light on the skin.

In an effort to transmit the light to the skin even more effectively, the massage means (31, 32, 33, 35, 36, 37, 37′, 38, 39 and 39′) can be transparent or translucent.

According to the examples depicted, the LEDs are built into the treatment head (6, 7, 8, 9) having a massage function in order to be as close to the skin as possible. However, it is completely feasible to arrange the LEDs in the body (2) of the apparatus, at the end where each treatment head (6, 7, 8, 9) is assembled, thereby simplifying the electrical connectors, which makes the apparatus safer, more economical and more compact.

Of course, the invention is in no way limited to the methods of implementation described and depicted, which have been provided only as examples. Modifications remain possible, particularly with regard to the makeup of the various components or by substituting equivalent techniques, while still remaining within the scope of protection of the invention.

1. An apparatus for treating skin, comprising:
a body comprising a casing forming a graspable area,
a means of supplying electrical power arranged in said body,
at least one treatment head,
at least one light source,
at least one light guide with a projection surface designed to receive light emitted by said at least one light source and to project at least one beam of light toward an area on the skin,
wherein said at least one light guide comprises an opposite surface with respect to said projection surface, said opposite surface comprising a means of distribution designed to distribute the light over said projection surface.

2. The apparatus according to claim 1, wherein said means of distribution comprises at least one surface that is at least partially reflective, designed to reflect part of the light toward said projection surface.

3. The apparatus according to claim 2, wherein said at least one light guide has at least one cross-section through which light can pass, which is a cross-section passing through a plane that is perpendicular to said projection surface, said cross-section being secant to the projection surface and said at least one surface following a first straight line and a second straight line, respectively.

4. The apparatus according to claim 3, wherein said at least one light source is arranged at one end of said cross-section between the first straight line and the second straight line.

5. The apparatus according to claim 4, wherein a distance between the first straight line and the second straight line is constant, as measured from the end where said at least one light source is arranged.

6. The apparatus according to claim 4, wherein a distance between the first straight line and the second straight line decreases, as measured from the end where said at least one light source is arranged.

7. The apparatus according to claim 5, wherein said means of distribution comprises at least two surfaces that are at least partially reflective, in which one of the two surfaces and said cross-section are secant along a third straight line, a distance between D1 and D2 being greater than a distance between D1 and D3.

8. The apparatus according to claim 3, wherein said light guide is a solid of revolution of said cross-section around a central axis.

9. The apparatus according to claim 8, wherein the opposite surface has flutes starting from said central axis.

10. The apparatus according to claim 5, wherein the opposite surface has hemispherical dimples.

11. The apparatus according to claim 1, wherein said opposite surface is at least partially covered with a white-colored layer.

12. The apparatus according to claim 1, wherein said opposite surface is at least partially covered with a metallic-colored layer.

13. The apparatus according to claim 1, wherein said projection surface has a means of diffusion.

14. The apparatus according to claim 1, wherein said at least one light source is a light-emitting diode.

15. The apparatus according to claim 1, wherein said at least one treatment head comprises a mechanical means of massage designed to come in contact with the skin, and means of maneuvering said massage means, powered by an electric motor connected to said means of electrical power supply.

16. The apparatus according to claim 15, wherein said massage means comprises at least one massage ball designed to be spun along at least one axis of vertical rotation that is perpendicular to the projection surface.

17. The apparatus according to claim 16, wherein said light guide has at least one through-hole through which said massage ball may pass.

18. The apparatus according to claim 8, wherein said light source comprises multiple light-emitting diodes distributed at regular intervals around said light guide.

19. The apparatus according to claim 15, wherein said means of massage comprises a support component designed to be placed against the face and two massage fingers designed to come into contact with the skin in an alternating manner.

20. The apparatus according to claim 19, wherein said light guide is arranged close to and roughly parallel with said support component.

21. The apparatus according to claim 20, wherein said light source comprises at least one light-emitting diode between said massage fingers and the body.

22. The apparatus according to claim 15, wherein said massage means comprises two massage rollers that can rotate around themselves along two axes of horizontal rotation that are parallel to one another and each one perpendicular to the axis of the body, a first roller comprising at least one paddle protruding radially from the surface of the first roller and the second roller having a smooth surface, said rollers being powered by a maneuvering means.

23. The apparatus according to claim 22, wherein it comprises at least one light guide designed to cover at least one contact area, at the end of the rollers that is designed to come into contact with the skin.

24. The apparatus according to claim 23, wherein said light source comprises at least one light-emitting diode near said contact area.

25. The apparatus according to claim 15, wherein said massage means comprises two cylindrical rollers that can rotate around themselves along two axes of rotation that are parallel to one another and with respect to the axis of the body.

26. The apparatus according to claim 25, wherein it comprises at least one light guide designed to cover at least one illumination area at one of the ends of the cylindrical rollers and/or a second illumination area situated between said cylindrical rollers, said first illumination area being designed to come into contact with the skin.

27. The apparatus according to claim 26, wherein said light source comprises at least one light-emitting diode near said first illumination area.

28. The apparatus according to claim 15, wherein said massage means is one of transparent or translucent.

29. The apparatus according to claim 1, wherein said at least one treatment head is detachable from said body.

30. The apparatus according to claim 29, comprising at least two different treatment heads that are interchangeable on the body.

31. The apparatus of claim 13, wherein the means of diffusion is texturing on said projection surface.
